(19) United States
(12) Patent Application Publication
(10) Pub. No.: US 2023/0398170 A1
(43) Pub. Date: Dec. 14, 2023

(54) A THERAPEUTIC OIL COMPOSITION AND A PROCESS FOR ITS PREPARATION

(71) Applicant: Mohan DEWAN, Pune (IN)

(72) Inventor: Mohan DEWAN, Pune (IN)

(21) Appl. No.: 18/030,372

(22) PCT Filed: Dec. 7, 2021
(86) PCT No.: PCT/IB2021/061399

§ 371 Date: Apr. 5, 2023

(30) Foreign Application Priority Data
Dec. 7, 2020 (IN) 202021053227

(51) Int. Cl. A61K 36/889; A61K 36/185; A61K 36/55; A61K 36/63; A61K 36/14; A61K 36/54; A61K 36/61; A61K 9/00; A61P 29/02; A61P 25/02

(52) U.S. Cl. CPC A61K 36/889; A61K 36/185; A61K 36/55; A61K 36/63; A61P 25/02; A61K 36/54; A61K 36/61; A61K 9/0014; A61P 29/02; A61K 36/14

(57) ABSTRACT

The present disclosure relates to a therapeutic oil composition and a process for its preparation. The therapeutic oil composition comprises at least one base oil selected from the group consisting of jojoba oil, flaxseed oil, olive oil, coconut oil; and additive oil mixture. The contents of each of the oil in the therapeutic oil composition are synergistically balanced to elicit complementary effects in the treatment of the human subjects.

### FIELD

The present disclosure relates to a therapeutic oil composition and a process for its preparation.

The biological material used in the present invention was purchased from various sellers listed on Amazon India.

### Definition

As used in the present disclosure, the following terms are generally intended to have the meaning as set forth below, except to the extent that the context in which they are used, indicate otherwise.

Manual Lymphatic Drainage (MLD) refers to a method of treatment which involves providing a light, skin-stretching massage that helps to promote the movement of lymphatic fluid out of the swollen limb.

Peripheral Neuropathy refers to a disorder that occurs when the peripheral nerves malfunction because of damaged or destroyed nerves.

### BACKGROUND

The background information hereinbelow relates to the present disclosure but is not necessarily prior art.

A lymphatic system is a crucial part of the human immune system. Through a network of hundreds of lymph nodes, the lymphatic system drains the fluid called lymph which is generally transported back into the bloodstream. The lymphatic system also removes bodily waste and carries white blood cells that help to prevent infection. When there is any kind of obstruction in the lymphatic system, fluid will start to build up. When the lymphatic system is blocked, it cannot carry out its pathogen-fighting activity and the fluid becomes stagnant, thereby impairing the immune system. In addition to the swelling and the immune system impairment, the stagnant lymph may cause on-site inflammation and infection. This stagnation may cause lymphedema, which can contribute to the long-term, serious, painful conditions such as cellulitis. To overcome the obstruction and for draining the fluid build-up, manual lymphatic drainage therapy is used. Lymphatic drainage treatments accelerate the absorption and transportation of lymphatic fluids which contain toxins, bacteria, viruses, and proteins.

The effectiveness of the lymphatic drainage treatments is enhanced when the therapy is coupled with therapeutic oils. Many of the essential oils such as sweet orange oil, lemon oil, mandarin oil, tangerine oil are used for lymphatic drainage treatments. The conventional therapeutic oils used in lymphatic drainage systems are processed at a high temperature which can damage the chemical structure of the oil and affect the therapeutic efficacy of the oil composition. Further, there is still room for developing an oil composition which can provide a better relief when compared to the conventional oil systems used for lymphatic drainage treatments.

Therefore, there is a need to develop a therapeutic oil composition.

### OBJECTS

Some of the objects of the present disclosure, which at least one embodiment herein satisfies, are as follows:

An object of the present disclosure is to ameliorate one or more problems of the prior art or to at least provide a useful alternative.

Another object of the present disclosure is to provide a therapeutic oil composition.

Yet another object of the present disclosure is to provide a therapeutic oil composition that can be used for lymphatic drainage treatment and pain relief.

Still another object of the present disclosure is to provide a therapeutic oil composition that can be used in the treatment of peripheral neuropathy.

Another object of the present disclosure is to provide a process for the preparation of a therapeutic oil composition.

Other objects and advantages of the present disclosure will be more apparent from the following description, which is not intended to limit the scope of the present disclosure.

### SUMMARY

The present disclosure relates to a therapeutic oil composition and a process for its preparation. In an aspect, the therapeutic oil composition comprises at least one base oil selected from the group consisting of jojoba oil, flaxseed oil, olive oil, and coconut oil in an amount in the range of 50% to 70% with respect to the total volume of the composition and additive oil mixture in an amount in the range of 30% to 50% with respect to the total volume of the composition; wherein the additive oil mixture comprises cypress oil, juniper oil, and cinnamon oil; wherein a volume ratio of the base oil to the additive oil mixture is in the range of 1:0.5 to 1:1.

In another aspect, the process for the preparation of a therapeutic oil composition comprises blending additive oils for a first predetermined time period to obtain an additive oil mixture. The additive oil mixture is allowed to stabilize for a second predetermined time period to obtain a homogeneous additive oil mixture. The so obtained homogeneous additive oil mixture is blended with at least one base oil for a third predetermined time period to obtain the resultant mixture. The resultant mixture is subjected to maturing by exposing it to sunlight for a fourth predetermined period followed by filtration to obtain the therapeutic oil composition.

### DETAILED DESCRIPTION

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

The lymphatic system protects the human body from illness-causing invaders, maintains body fluid levels, absorbs digestive tract fats, and removes cellular waste. When there is any kind of obstruction in the lymphatic system, fluid can start to build up. When the lymphatic system is blocked, it cannot carry out its functions and the fluid becomes stagnant, thus impairing the immune system. The lymphatic blockages can be cleared by the use of lymphatic drainage treatment which includes the use of therapeutic oils.

The present disclosure provides a therapeutic oil composition and a process for its preparation.

In an aspect of the present disclosure, there is provided a therapeutic oil composition.

The therapeutic oil composition comprises at least one base oil selected from the group consisting of jojoba oil, flaxseed oil, olive oil, and coconut oil in an amount in the range of 50% to 70% with respect to the total volume of the composition; and additive oil mixture in an amount in the range of 30% to 50% with respect to the total volume of the composition, wherein the additive oil mixture comprises cypress oil, juniper oil, and cinnamon oil; wherein a volume ratio of the base oil to the additive oil mixture is in the range of 1:0.5 to 1:1.

In an exemplary embodiment of the present disclosure, the volume ratio of the base oil to the additive oil mixture is in the range of 1:0.54. In another exemplary embodiment of the present disclosure, the volume ratio of the base oil to the additive oil mixture is in the range of 1:0.5.

In an embodiment of the present disclosure, the volume ratio of the cypress oil to the juniper oil to the cinnamon oil is in the range of 1:1:1.

In an embodiment of the present disclosure, the additive oil mixture optionally comprises eucalyptus oil.

In an embodiment of the present disclosure, the volume ratio of the cypress oil to the juniper oil to the cinnamon oil to the eucalyptus oil is in the range of 1:1:1:0.5.

Jojoba oil is a liquid produced from the seeds of Simmonds chinensis plant, a shrub, which is native to southern Arizona, southern California, and northwestern Mexico. The jojoba seeds comprise approximately 50% of the oil by weight. The terms “jojoba oil” and “jojoba wax” are often used interchangeably because the wax visually appears to be a mobile oil, but as a wax, it is composed almost entirely (˜97%) of mono-esters of long-chain fatty acids and alcohols (wax ester), accompanied by only a tiny fraction of triglyceride esters. Due to its composition jojoba oil/wax has extreme shelf-life stability and extraordinary resistance to high temperatures when compared with true vegetable oils.

Flaxseed oil is a colourless to yellowish oil obtained from dried, ripened seeds of Linum usitatissimum. It is also known as linseed oil or flax oil. The flaxseed oil contains both omega-3 fatty acids such as α-linolenic acid (ALA) and omega-6 fatty acids.

Olive oil is extracted from the fleshy part of the ripened fruit of the olive tree i.e. Olea europaea. The olive oil is predominantly produced in the countries of the Mediterranean Basin. The oil is also produced in California, South America, and Australia. The key content of the olive oil is oleic acid, with smaller amounts of fatty acids.

Coconut oil is extracted from the kernel of matured coconuts of Cocos nucifera. Coconut oil contains high amounts of saturated fats. The virgin coconut oil has anti-inflammatory, skin protective, and antibacterial properties that can help to treat infections. The coconut oil is also used as an edible oil.

Cypress oil derives from the cypress tree (Cupressus Sempervirens), native to Asian and Mediterranean countries. Cypress oil is obtained from the twigs, stems, and leaves of the cypress tree. Cypress oil has many health benefits, as it has antibacterial, antimicrobial, and antifungal properties.

Juniper oil is derived from the berry-like seed cones of Juniperus communis. Juniper has been an important spice in numerous European cuisines, and particularly where it grows abundantly such as throughout the Alpine regions. Traditionally, juniper berries are steam-distilled to produce the oil that may vary from colorless to yellow or pale green. Some of its chemical components are terpenoids and aromatic compounds, such as cadinene, and sesquiterpene.

Cinnamon oil is derived from the bark or leaves of several types of trees, including the Cinnamomum verum and the Cinnamomum cassia. Most commercially available cinnamon oil is derived from the Cinnamomum cassia tree and is called cassia cinnamon.

Eucalyptus oil is derived from the leaves of Eucalyptus globulus. The leaves are dried, crushed, and distilled to extract the eucalyptus oil. The eucalyptus trees are native to Australia but are being cultivated across the globe due to their medicinal properties.

In an embodiment of the present disclosure, the base oils and the additive oils are obtained by cold pressing.

Cold Pressing is a mechanical process in which the oil is extracted and separated from the seeds or nuts without chemical intervention and without the addition of any substances at a temperature that does not exceed 35° C. No external heat is used in this process. Extracting oil through cold pressing involves crushing the seed or nut and forcing out the oil.

The process of the cold pressing is advantageous due to the fact that the activity and the functional groups (chemical constituents) of the oil are intact and oil can be obtained undamaged.

In an embodiment of the present disclosure, the therapeutic oil composition is used for manual lymphatic drainage, pain relief, and peripheral neuropathy.

In another aspect of the present disclosure, there is provided a process for the preparation of the therapeutic oil composition.

The process is described in detail.

In a first step, additive oils are blended for a first predetermined time period to obtain an additive oil mixture.

In an embodiment of the present disclosure, the additive oils comprise cypress oil, juniper oil, and cinnamon oil.

In an embodiment of the present disclosure, the additive oils cypress oil, juniper oil, and cinnamon oil are blended in a volume ratio of 1:1:1.

In an embodiment of the present disclosure, the additive oil mixture comprises a predetermined amount of eucalyptus oil.

In an embodiment of the present disclosure, the additive oil mixture comprises cypress oil, juniper oil, cinnamon oil, and eucalyptus oil blended in a volume ratio of 1:1:1:0.5.

In an embodiment of the present disclosure, the first predetermined time period is in the range of 10 minutes to 30 minutes. In an exemplary embodiment, the first predetermined time period is 15 minutes. In another exemplary embodiment, the first predetermined time period is 20 minutes.

In a second step, the additive oil mixture is stabilized for a second predetermined time period to obtain a homogeneous additive oil mixture.

In an embodiment of the present disclosure, the second predetermined time period is in the range of 10 minutes to 30 minutes. In an exemplary embodiment, the second predetermined time period is 20 minutes. In another exemplary embodiment, the second predetermined time period is 15 minutes.

In the stabilization process, the additive oil mixture is placed at room temperature that allow forming a homogeneous additive oil mixture. The process also ensures to identify the stability issues with the oil mixture i.e. phase separation/cracking and the like.

In a third step, the homogeneous additive oil mixture is blended with at least one base oil for a third predetermined time period to obtain the resultant mixture.

In an embodiment of the present disclosure, the third predetermined time period is in the range of 10 minutes to 30 minutes. In an exemplary embodiment, the third predetermined time period is 20 minutes.

In an embodiment of the present disclosure, the base oil is subjected to heating at a temperature in the range of 20° C. to 30° C. before blending with the homogeneous additive oil mixture. In an exemplary embodiment, the temperature is 25° C.

The heat treatment at 20° C. to 30° C. allows viscous base oils to miscible with the homogeneous additive oil mixture uniformly.

In a final step, the resultant mixture is subjected to maturing by exposing it to sunlight for a fourth predetermined period followed by filtration to obtain the therapeutic oil composition.

In an embodiment of the present disclosure, the fourth predetermined time period is in the range of 5 days to 20 days. In an exemplary embodiment, the fourth predetermined time period is 15 days.

In an embodiment of the present disclosure, the resultant mixture is placed in sunlight in a transparent container for optimum absorption of sunlight.

In an embodiment of the present disclosure, following filtration the therapeutic oil composition is stored in amber coloured bottles and stored in a cool and dry place.

The process of exposing the resultant mixture to sunlight allows the process of maturation of organic matter in the resultant mixture, thus providing the therapeutic oil composition effective for manual lymphatic drainage, pain relief, and peripheral neuropathy.

The foregoing description of the embodiments has been provided for purposes of illustration and not intended to limit the scope of the present disclosure. Individual components of a particular embodiment are generally not limited to that particular embodiment but are interchangeable. Such variations are not to be regarded as a departure from the present disclosure, and all such modifications are considered to be within the scope of the present disclosure.

The present disclosure is further described in light of the following experiments which are set forth for illustration purposes only and not to be construed for limiting the scope of the disclosure. The following experiments can be scaled up to industrial/commercial scale and the results obtained can be extrapolated to industrial scale.

### EXPERIMENTAL DETAILS

### Example 1

1200 ml of cypress oil, 1200 ml of juniper oil, and 1200 ml of cinnamon oil were intimately blended for 15 min to obtain an additive oil mixture. The additive oil mixture was allowed to stabilize for 20 min at room temperature to obtain a homogeneous additive oil mixture. The so obtained homogeneous additive oil mixture was blended with 6600 ml of jojoba oil for 20 min to obtain the resultant mixture. The resultant mixture was placed in a transparent container, exposed to sunlight for 15 days followed by filtration to obtain the therapeutic oil composition. The so obtained therapeutic oil composition was stored in amber coloured bottles in a cool and dry place.

### Example 2

2 lit of cypress oil, 2 lit of juniper oil, 2 lit of cinnamon oil, and 1 lit of eucalyptus oil were intimately blended for 20 min to obtain an additive oil mixture. The additive oil mixture was allowed to stabilize for 15 min at room temperature to obtain a homogeneous additive oil mixture. Separately, 14 lit of coconut oil was heated to 25° C. to obtain a warm coconut oil. The so obtained homogeneous additive oil mixture was blended with 14 lit of the warm coconut oil for 20 min to obtain the resultant mixture. The resultant mixture was placed in a transparent container, exposed to sunlight for 15 days followed by filtration to obtain the therapeutic oil composition. The so obtained therapeutic oil composition was stored in amber coloured bottles in a cool and dry place.

### Anecdotal Studies:

Studies were carried out to determine the efficacy of therapeutic oil composition obtained in examples 1 and 2 against various conditions such as a severe sprain, peripheral neuropathy, rheumatoid arthritis, arthritis hip, and the like. The therapeutic oil composition was applied in the desired amount to the affected body parts. All the subjects participating in the study have prior used various commercially available medications such as pain-relieving ointments, gels, oils, and the like. But the subjects were not relieved from the symptoms.

Further, the subjects used the therapeutic oil composition of the present disclosure, the results obtained are summarized below as anecdotal studies.

### Trial 1:

A 55-year-old male subject presented with a severe sprain of the ankle and was unable to walk normally. The subject applied 15 ml of the therapeutic oil composition of Example 1 by rubbing at the affected site till the oil was absorbed completely into the skin. The therapeutic oil composition was applied twice daily in the morning and night before going to bed. After continuing the therapy for a period of 10 days the subject was relieved from the pain and able to walk normally.

### Trial 2:

A 59-year-old female subject with a history of diabetes from 10 years presented with peripheral neuropathy with symptoms of loss of sensation in the hands/fingers and foot for 6 months. The subject applied the therapeutic oil composition of Example 1 by rubbing at the affected site till the oil was absorbed completely into the underlying tissues. The therapeutic oil composition was applied twice daily in the morning and night before going to bed. After continuing the therapy for a period of 30 days the subject was able to regain some sensation in hands/fingers and the foot.

Trial 3:

A 65-year-old female subject presented with rheumatoid arthritis, the knee was painful and there was morning stiffness. The subject had tried various commercial remedies, but there was no relief. The subject applied the therapeutic oil composition of Example 2 by rubbing on the affected site till the oil was absorbed completely into the skin. The therapeutic oil composition was applied twice daily in the morning and night before going to bed. After continuing the therapy for a period of 15 days the subject obtained significant relief from the pain and the stiffness of the knee was also reduced.

Trial 4:

A 40-year-old male subject presented with arthritis of the left hip with symptoms of difficulty in walking, pain in the groin area and outer thigh. The subject applied the therapeutic oil composition of Example 1 by rubbing at the affected site till the oil was absorbed completely into the skin. The therapeutic oil composition was applied thrice daily i.e. after bath, in the afternoon, and before going to bed. After continuing the therapy for a period of 30 days the subject had significant relief from the symptoms.

It is evident from the above examples and anecdotal studies that the therapeutic oil composition with the blend of the base oil and additive oil mixture synergistically provides relief to the subjects from the symptoms. Further, additional anecdotal studies are in progress.

### TECHNICAL ADVANCEMENTS

The present disclosure described hereinabove has several technical advantages including, but not limited to, the realization of a therapeutic oil composition:
that is used for the manual lymphatic drainage treatment, pain relief, and peripheral neuropathy;
is safe and effective;
can be used concurrently with other medicines;
   and
a process for preparing a therapeutic oil composition which is simple and economical.

Throughout this specification, the word “comprise”, or variations such as “comprises” or “comprising, will be understood to imply the inclusion of a stated element, integer or step,” or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

The numerical values given for various physical parameters, dimensions and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A therapeutic oil composition comprising:
a. at least one base oil selected from the group consisting of jojoba oil, flaxseed oil, olive oil, and coconut oil in an amount in the range of 50% to 70% with respect to the total volume of the composition; and
b. additive oil mixture in an amount in the range of 30% to 50% with respect to the total volume of the composition;
wherein said additive oil mixture comprises cypress oil, juniper oil, and cinnamon oil;
wherein a volume ratio of said base oil to said additive oil mixture is in the range of 1:0.5 to 1:1.

2. The composition as claimed in claim 1, wherein said additive oil mixture comprises a predetermined amount of eucalyptus oil.

3. The composition as claimed in claim 1, wherein a volume ratio of said cypress oil to said juniper oil to said cinnamon oil is 1:1:1.

4. The composition as claimed in claim 1, wherein a volume ratio of said cypress oil to said juniper oil to said cinnamon oil to said eucalyptus oil is in the range of 1:1:1:0.5.

5. The composition as claimed in claim 1, wherein said composition is used for manual lymphatic drainage, pain relief, and peripheral neuropathy.

6. A process for the preparation of a therapeutic oil composition as claimed in claim 1, said process comprising the following steps:
a. blending additive oils for a first predetermined time period to obtain an additive oil mixture;
b. stabilizing said additive oil mixture for a second predetermined time period to obtain a homogeneous additive oil mixture;
c. blending said homogeneous additive oil mixture with at least one base oil for a third predetermined time period to obtain the resultant mixture; and
d. maturing said resultant mixture by exposing it to sunlight for a fourth predetermined time period followed by filtration to obtain the therapeutic oil composition.

7. The process as claimed in claim 6, wherein said base oil is subjected to heating at a temperature in the range of 20° C. to 30° C. before blending with the homogeneous additive oil mixture.

8. The process as claimed in claim 6, wherein said first, said second and said third predetermined time period is in the range of 10 minutes to 30 minutes.

9. The process as claimed in claim 6, wherein said fourth predetermined time period is in the range of 5 days to 20 days.

10. The composition as claimed in claim 2, wherein a volume ratio of said cypress oil to said juniper oil to said cinnamon oil to said eucalyptus oil is in the range of 1:1:1:0.5.
